(19) United States
(12) Patent Application Publication
(10) Pub. No.: US 2021/0259564 A1
(43) Pub. Date: Aug. 26, 2021

(54) Health Monitoring Device

(71) Applicant: Stacey Watt, Grand Island, NY (US)

(72) Inventor: Stacey Watt, Grand Island, NY (US)

(21) Appl. No.: 17/178,366

(22) Filed: Feb. 18, 2021

Related U.S. Application Data
(60) Provisional application No. 62/978,913, filed on Feb. 20, 2020.

(51) Int. Cl. A61B 5/0205; A61B 5/00; A61B 5/145

(52) U.S. Cl. CPC A61B 5/02055; A61B 5/7455; A61B 2560/0271; A61B 5/14532; A61B 2503/10; A61B 5/6833

(57) ABSTRACT

This invention is directed to a wearable device that is capable of monitoring the health data of an individual, in particular, of an athlete while training. The device is also capable of providing location information in the event that the individual has a health issue while training. The device is also capable of receiving haptic signals from an external device. The device comprises conductive adhesive hydrogel, a battery, a GPS system, a temperature sensor, a health sensor, a storage memory, a haptic, and a means of communication.

### CROSS-REFERENCE TO RELATED APPLICATIONS

This application claims priority to U.S. Provisional Application having Ser. No. 62/978,913, filed on Feb. 20, 2020, the entire disclosure of which is hereby incorporated herein by reference.

### FIELD OF INVENTION

This invention is directed to a wearable device that is capable of continuously or periodically monitoring the health data of an individual, in particular, of an athlete while training. The device is also capable of receiving and conveying signals, including receiving signals that instruct the athlete to return from training. Importantly, the device is capable of providing location information in the event that the athlete has a health issue while training.

### BACKGROUND OF INVENTION

When athletes are training under the guidance of a coach, often the coach isn't actually with the athlete. For example, if a coach is training a runner, the runner will be out on the track or at some other location and the coach may be at the training facility.

When the athlete and coach are separated, it is desirable for the coach to have real-time information about the health profile of the athlete. The health profile may include information about heart rate, blood pressure, body temperature, metabolic rate, glucose levels, oxygen levels, pulse-ox, and other information.

In the event that the athlete is having a health issue while training, it would be desirable for the coach to be alerted. It would also be desirable for the coach to know the location of the athlete when this is happening. For example, if an athlete is training in very cold weather, the coach would want to locate the individual as soon as possible in the event that the athlete is experiencing a health or safety issue.

From the standpoint of the individual, the device needs to be as unobtrusive as possible, while providing a variety of different health data.

While there are wearable health monitoring devices on the market, none of them are set up to notify a coach or someone else about a health or safety issue that an individual is having while training. There is also nothing on the market that allows a coach to monitor the performance of the athlete and to signal to the athlete that he or she should return to the training facility because the coach has identified a health concern to the athlete. It would also be desirable for the arch to monitor the athlete's performance while the athlete is training. This can provide information on adjusting he athlete's training for better performance. In some instances, if an athlete becomes lost or if the health monitoring device becomes detached, it would be desirable for the coach to have last known location information for the athlete.

Thus, there is a need for an unobtrusive, wearable health monitoring device for use by an individual that provides health and location information to another individual.

### SUMMARY OF THE INVENTION

Accordingly, it is the subject of this invention to provide an unobtrusive, wireless, wearable health monitoring device for athletes to use while training. The wearable health monitoring device may provide continuous or periodic health monitoring.

In one embodiment, the device is comprised of: a conductive adhesive hydrogel, a monitor assembly that includes a battery, a global position system (GPS) component, a temperature sensor, a health sensor, and a means of communication.

The monitor assembly further comprises one or more health sensors encased in a housing and a means for attaching the housing to the body. The housing and one or more sensors in the housing are placed proximate to or against the skin surface of an individual.

The health sensor may be an infrared sensor, thermistor, pulse oximeter, EKG monitor, cardiac telemetry monitor, blood pressure monitor, heart rate monitor, respiration rate monitor, body temperature monitor, accelerometer and/or an electrocardiogram monitor. In some embodiments, the sensor may transmit a signal to a processor such as a CPU, microprocessor or microcontroller contained within the sensor housing.

The health sensor measurement is then wirelessly transmitted to a remote display device such as a computer, personal digital assistant, mobile device or tablet. The sensor may transmit the signal directly to the remote display device or send it through other processes in the sensor housing unit. The transmission may be through internet, an intranet, private or public networks, or the cloud. The cloud includes any internet based computing system where different servers, storage and applications, receive the data and then send it on to the remote display device.

In some embodiments, the remote display device may track physiological signal trends, activate an alarm when pre-determined parameters are exceeded, display the vital signs or other physiological signals, display trends in physiological signals

The remote display device may have an application that allows it to track vital sign trends or other physiological signals, activate an alarm when pre-determined parameters are exceeded, and/or display the physiological signals or trends in physiological signals.

In another embodiment, sensor readings that exceed pre-set parameters may trigger a message sent to the remote display device, which may cause the reader of the device to alert emergency services.

In a preferred embodiment, a health monitoring device is provided, which comprises: a housing having a top surface and bottom surface; an adhesive layer adhered to the bottom surface of the housing; a conductive adhesive hydrogel layer, which is adhered to the adhesive layer; a battery capable of powering the health monitoring device and contained within the conductive adhesive hydrogel layer; a GPS system capable of providing GPS information and contained within the conductive adhesive hydrogel layer; a temperature sensor capable of measuring temperature information and contained within the conductive adhesive hydrogel layer; at least one health sensor capable of measuring physiological signals and contained within the conductive adhesive hydrogel layer; a processor capable of storing and processing information and contained within the conductive adhesive hydrogel layer, wherein the processor is in communication with the GPS system, the temperature sensor, and the at least one health sensor; a storage memory contained within the conductive adhesive hydrogel layer, wherein the storage memory is in communication with the processor; a communication chip capable of communicating with an external device and contained within the conductive adhesive hydrogel layer, wherein the communication chip is in communication with the storage memory; and, a haptic capable of signaling a user of the health monitoring device and contained within the conductive adhesive hydrogel layer.

In another preferred embodiment of the invention, a remote display device is provided, which comprises: a display that is capable of displaying all of the health information that it receives from a health monitoring device; a processor that is capable of running an application for processing physiological signals received externally; a communication chip capable of communicating with an external device; and, a haptic singular that is capable of communicating with an external haptic.

In another preferred embodiment, a system for monitoring the training of an athlete is provided wherein the system includes both a health monitoring device as described above and a remote display device as described above.

### BRIEF DESCRIPTION OF THE DRAWINGS

FIG. 1 depicts a top view of the device.

FIG. 2 depicts a bottom view of the device.

FIG. 3 depicts a cross section of the device.

FIG. 4 depicts an exploded view of the components of the device.

FIG. 5 depicts a battery charging station having several battery chargers.

FIG. 6 depicts a remote display device.

### DETAILED DESCRIPTION OF THE INVENTION

### Components List

health monitoring device—10
printed indicia—12
battery—20
housing—30
adhesive layer—32
conductive adhesive hydrogel—34
GPS system—36
temperature sensor—38
health sensor—40
storage memory—42
communication chip—44
processor—46
haptic—48
bottom layer 50
battery charging station—100
battery charger—110
remote display device—200
display—205
processor—210
haptic signaler—215
communication chip—220

Provided herein is a means for continuous, non-invasive, real-time wireless monitoring of one or more of an individual's physiological signals in an athletic training setting.

The system described herein may be used to continuously monitor one or more vital signs such as the four primary vital signs: body temperature, blood pressure, heart rate, and respiratory rate; as well as additional physiological signals.

The physiological signals are measured using a system comprising a reusable sensor housing, at least one, two, three, four, or more reusable physiological sensors, a user replaceable battery, wherein the battery may be rechargeable or non-rechargeable, a power button or other means for activating the device such as through an application located on the remote display device, a microcontroller, a power controller, a communication chip, a disposable means for attachment. The sensor in the sensor housing may measure physiological signals including, but not limited to, temperature, blood pressure, oxygen levels, electrical conduction, pulse, respiration, heart rate and rhythm, activity levels and the like which may be referred to herein as physiological signs. The sensor and the sensor housing are unable to display any physiological data without the use of another separate, additional device such as the remote display device. The sensor or sensor housing unit transmits data and communicates with the remote display device through wireless communication.

The term “sensor” as used herein refers to any component that is capable of detecting physiological changes through the skin of an individual. Sensors may include any type of electrical, optical, mechanical, and/or chemical non-invasive sensors. The sensor may be any type of sensor useful in continuous monitoring, including, but not limited to, an infrared sensor, thermistor, pulse oximeter, accelerometer, EKG monitor, cardiac telemetry monitor, blood pressure monitor, heart rate monitor, respiration rate monitor, body temperature monitor, electrocardiogram monitor and the like.

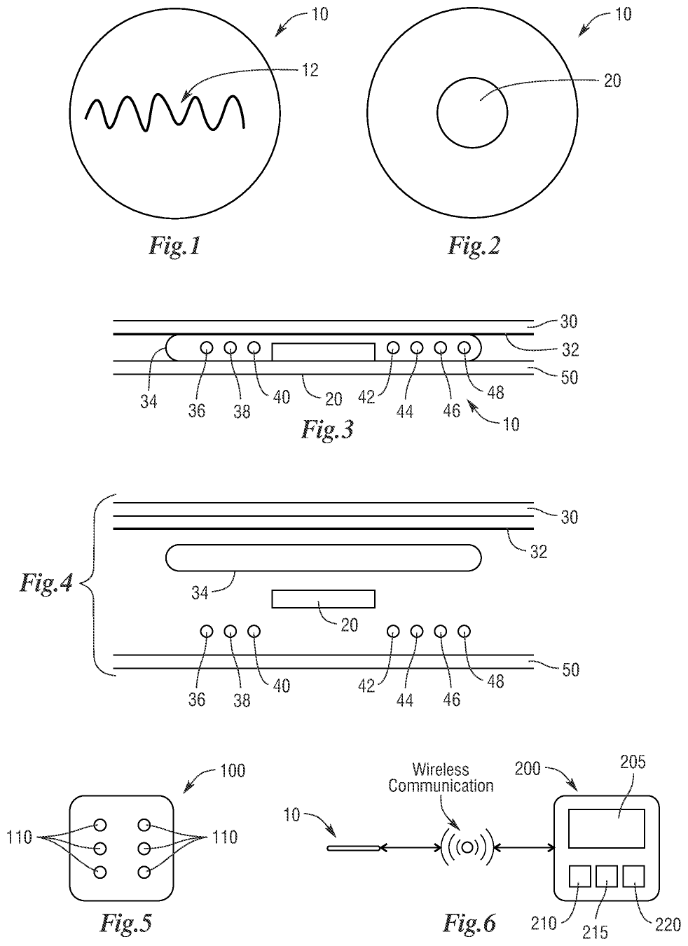

FIG. 1 depicts a top view of the health monitoring device 10, wherein there may be printed indicia 12 on the health monitoring device 10. The printed indicia 12 may be a logo of the team that the individual is training under or may be some other logo or any type of printed indicia.

FIG. 2 depicts a bottom view of the health monitoring device, wherein a battery 20 can be seen.

In one embodiment, as depicted in FIGS. 3 and 4, the health monitoring device 10 is comprised of: a housing 30, an adhesive layer 32, a conductive adhesive hydrogel 34, a battery 20, a GPS system 36, a temperature sensor 38, a health sensor 40, a storage memory 42, a communication chip 44, a processor 46, and haptic 48. In some embodiments, bottom layer 50 encloses the conductive hydrogel layer 34. In other embodiments bottom layer 50 is not necessary.

In a preferred embodiment, the GPS system 36, temperature sensor 38, and health sensor 40, are all in communication with storage memory 42, communication chip 44, and processor 46. In another preferred embodiment, health sensor 40 may be made up of several different sensors (not shown) to monitor heart rate, blood pressure, pulse-ox, metabolic rate, glucose levels, oxygen levels, and other health information. In an embodiment, the health monitor device 10 does not include a processor 46. In this embodiment, the health monitor device 10 sends raw health data to a remote device.

In one embodiment, the battery 20 is rechargeable or disposable. In a preferred embodiment, battery 20 can be recharged by battery charger 110 at battery charging station 100.

In one embodiment, housing 30 is made of any suitable material such as plastic or a flexible plastic or rubber material. Adhesive layer 32 is any adhesive that effectively attaches the housing to the conductive adhesive hydrogel 34 and in some cases adhesive layer 32 is not necessary and conductive adhesive hydrogel 34 is sufficient for adhesion.

In preferred embodiments, the GPS system 36, temperature sensor 38, health sensor 40, storage memory 42, communication chip 44, processor 46, and haptic 48 are all contained within conductive adhesive hydrogel 34. Conductive adhesive hydrogel 34 can be placed directly on the skin, preferably, behind the ear.

In some embodiments, the storage memory 42 and processor 46 may be replaced by a microcontroller. The microcontroller may include a CPU storage/memory (e.g., RAM, ROM, EEPROM, flash), general purpose input/output (GPIO), analog-to-digital (A/D) and digital-to-analog (D/A) converters, as well as digital signal processors (DSP). The processor or microcontroller is capable of controlling the measuring sequence, signal processing, and calculation of physiological parameters from monitoring data.

In another embodiment, as depicted in FIG. 5, the battery 20 may be charged by a battery charging station 100 having several battery chargers 110 or a back-up battery. The battery 20 is sealed with plastic and in one embodiment, the battery is magnetized.

The conductive adhesive hydrogel 34 allows the health monitoring device 10 to be placed behind the ear (not shown) of an individual or athlete that is training. In this way, the health monitoring device 10 is unobtrusive to the individual or athlete. Additionally, there is a lot of vasculature around the neck and back of the ear, but this area of the body does not have a lot of sweat, so the health monitoring device 10 will not have extensive interference from sweat. Another benefit of this location is that the health monitor device 10 will not be noticeable on the athlete.

In another embodiment, the health monitoring device 10 is water-proof and reusable and approximately 5 mm in thickness. In this embodiment, the adhesive layer 32 that connects the health monitoring device 10 to the athlete or user is disposable, but the health monitoring device components are is reusable. The health monitoring device 10 is also customizable with respect to size.

The sensor unit may be attached to the body by any means generally used including disposable adhesive patches. The health monitoring device 10 may include unique identification such as specific bar code type sensor information for each sensor.

In some embodiments, the temperature sensor 38 may be a thermistor. A thermistor is a temperature-sensing element composed of sintered semiconductor material which exhibits a large change in resistance proportional to a small change in temperature. In some embodiments the thermistor measures core temperature. In other embodiments, the thermistor measures skin temperature. In additional embodiments, the health sensor 40 is an infrared sensor, pulse oximeter, EKG monitor, cardiac telemetry monitor, accelerometer, blood pressure monitor, heart rate monitor, respiration rate monitor and the like.

As depicted in FIG. 6, health monitoring device 10 is in communication with remote display device 200 by way of wireless communication. Wireless communication may include, but is not limited to satellite communication, radar, mobile telephone system or cellular communication, global positioning system, infrared communication, WiFi, blue tooth, and television and radio broadcasting.

Remote display device 200 includes display 205 that is capable of displaying all of the health information that it receives from health monitoring device 10. In addition, remote display device 200 includes a processor 210. Processor 210 runs applications for analyzing data. The applications include pre-set parameters for health monitoring warnings.

For example, when the athlete's heart rate measures too high, the application will alert the coach of the remote display device 200 of this health issue. Parameters will also be set for blood pressure, pulse-ox, metabolic rate, glucose levels, oxygen levels, and any other health information that may be supplied by health monitoring device 10.

The remote display device 200 may include a software program that can be launched from a computer-readable medium in a computer-based system to execute the functions defined in the software program. One of ordinary skill in the art will further understand the various programming languages that may be used. For example, the programs can be an object-orientated format using an object-oriented language, such as Java, C++, or one or more other languages. Alternatively, the programs can be a procedure-orientated format using a procedural language, such as assembly, C, etc. The software components can communicate using any of a number of mechanisms well known to those of ordinary skill in the art, such as application program interfaces or interprocess communication techniques, including remote procedure calls or others. The teachings of various embodiments are not limited to any particular programming language or environment.

Remote display device 200 also includes haptic signaler 215, which communicates to haptic 48 of health monitor device 10. Remote display device 200 sends and receives information by way of communication chip 220. In one embodiment, if processor 210 indicates that the health parameters of the athlete are not within the set limits, it will cause haptic signaler 215 to signal haptic 48 of health monitor device 10 to alert the athlete that his or her health indicators are at dangerous levels and that the athlete should return to the training facility. In another embodiment, the coach is able to instruct remote display device 200 to cause haptic signaler 215 to indicate to signal haptic of health monitor device 10 that the athlete is to return to the training facility. This may be necessary if the coach would like to set up an impromptu meeting with one or more athletes.

In one embodiment, haptic 48 refers to any technology that can create an experience of touch by applying forces, vibrations, or motions to the user. In use, haptic signaler 215 of remote display device 200 will signal to haptic 48 to initiate a haptic sensation to the athlete. When the athlete notices the haptic sensation produced by haptic 48, the athlete will know to either return to the training facility or otherwise communicate with the coach or person that sent the haptic signal.

It is noted that while a haptic 48 and haptic signaler 215 have been described, another means for alerting the athlete can be imagined. For example, an audible sound or visible light may be used to alert the athlete.

The present invention offers many benefits, including, among others, providing geographic tracking of an athlete to a coach. In this way, the coach can keep a close watch on all of his or her athletes. Often triathletes train by themselves. When a triathlete is out lake swimming, running, or biking, a coach having geographical location information about the triathlete can ensure their safety in the event of an injury or some other situation. In one embodiment, the geographic location of an athlete will be relayed to the coach to make sure that the athlete hasn't veered off course or become lost or stuck in inclement weather. Importantly, if the health device becomes detached from the athlete, it will communicate to the remote display device that it is no longer attached. The coach can use this last known geographic location of the health monitor device to start looking for the athlete.

The present invention also offers additional benefits such as allowing a coach to track the health data of an athlete and providing a training profile of the athlete to the coach. In another embodiment, the health data includes heart rate, blood pressure, body temperature, metabolic rate, glucose levels, oxygen levels, pulse-ox, and other information. In another embodiment, the training profile of the athlete includes previous speeds and times and distance of an athletic activity so that the coach and athlete may compare results. Overall, the coach will be able to assess the training efficacy of the athlete with this information.

In another embodiment, the health monitoring device 10 may have thresholds set so that once a certain blood pressure, body temperature, or heart rate is reached, an alert will be sent to the coach. The thresholds may include maximums and minimums. For example, if the body temperature is over a certain temperature, the alert will be sent or if the body temperature drops below a certain temperature, the alert will be sent.

In another embodiment, there is a detachment alert such that the coach will be alerted if the health monitoring device 10 becomes detached from the individual. In another embodiment, haptic 48 of health monitoring device 10 is capable of issuing an audible alert if the athlete believes that he or she is in an unsafe situation. This will alarm anyone nearby that something is wrong, while also alerting the coach that something is wrong.

In a further embodiment, the communication chip 44 of health monitoring device 10 is two-way. That is, communication chip 44 communicates to the remote display device 200 and the coach can also signal the health monitoring device 10 to signal to the individual (by way of haptic signer 215) if the coach is concerned about the vital signs of the individual or the coach needs to get in touch with the individual and needs them to contact the coach or return back to their training center or facility.

As can be imagined, the health monitoring device 10 may also supply the athlete's training information to the remote display device 200. For example, it may show at what point during the training the athlete was hitting his or her peak performance.

### Example

In use, it is envisioned the an athlete at a training facility will obtain a fully charged health monitor device 10 from battery charging station 100. The athlete will attach health monitoring device 10 behind his or her ear before heading out to practice or train.

As the athlete is training, the temperature sensor 38 and health sensor 40 will continuously monitor temperature and other health indicators. Preferably, the health sensor 40 will be able to collect blood pressure, oxygen levels, electrical conduction, pulse, respiration, heart rate, heart rhythm, activity levels, metabolic rate, glucose levels, and pulse-ox data. It is noted that more than one health sensor 40 might be required in order to collect all of the health data. The health data is stored in storage memory 42 and then sent to processor 46 for processing and then sent back to the storage memory 42 for transmission to the remote display device 200. Alternatively, the health data may be stored in processor 46 and then processor and sent to he storage memory 42. Communication chip 44 is capable of sending and receiving information. Initially, the communication chip 44 sends the health data from either the processor 46 or storage memory 42 to the remote display device 200. The health data may be sent as raw data or processed date.

The coach will have constant access to remote display device 200 which will continuously display the health data or physiological signals that are sent from communication chip 44 of health monitoring device 10 to communication chip 220 of remote display device. The data will be displayed on display 205 of remote display device 200. The remote display device 200 will have the capability to process raw or already processed data in processor 210. If the display 205 of remote display device 200 indicates that the health data or physiological signals of the athlete are outside certain parameters or if the coach notices this, then the coach can instruct haptic signaler 215 of remote display device 200 to signal haptic 48 of health monitoring device 10 to indicate to the athlete to return from training. Remote display device 200 may also be programmed to automatically have haptic signaler 215 signal to haptic 48 once certain parameter thresholds are reached. The signal from haptic signaler 215 to haptic 48 are sent by communication chip 220 of remote display device 200 to communication chip 44 of health monitoring device 10. In the event that health monitor device 10 falls off of the individual or athlete, the coach will have the last known location of the individual or athlete.

It will be appreciated by those skilled in the art that while the health date monitoring device has been described in detail herein, the invention is not necessarily so limited and other examples, embodiments, uses, modifications, and departures from the embodiments, examples, uses, and modifications may be made without departing from the process and all such embodiments are intended to be within the scope and spirit of the appended claim

What is claimed is:

1. A health monitoring device comprising:
a housing having a top surface and bottom surface;
an adhesive layer adhered to the bottom surface of the housing;
a conductive adhesive hydrogel layer, which is adhered to the adhesive layer;
a battery capable of powering the health monitoring device and contained within the conductive adhesive hydrogel layer;
a GPS system capable of providing GPS information and contained within the conductive adhesive hydrogel layer;
a temperature sensor capable of measuring temperature information and contained within the conductive adhesive hydrogel layer;
at least one health sensor capable of measuring physiological signals and contained within the conductive adhesive hydrogel layer;
a processor capable of processing and storing information and contained within the conductive adhesive hydrogel layer, wherein the processor is in communication with the GPS system, the temperature sensor, and the at least one health sensor;
a storage memory contained within the conductive adhesive hydrogel layer, wherein the storage memory is in communication with the processor;
a communication chip capable of communicating with an external device and contained within the conductive adhesive hydrogel layer, wherein the communication chip is in communication with the storage memory; and,
a haptic capable of signaling a user of the health monitoring device and contained within the conductive adhesive hydrogel layer.

2. The device of claim 1 further comprising a bottom layer below the conductive adhesive hydrogel layer and wherein the bottom layer encloses health monitor device and includes an adhesive layer for attachment.

3. The device of claim 1 wherein the battery is rechargeable.

4. The device of claim 1 wherein the at least one health sensor measures at least one of the following physiological signals: blood pressure, oxygen levels, electrical conduction, pulse, respiration, heart rate, heart rhythm, activity levels, metabolic rate, glucose levels, and pulse-ox.

5. The device of claim 4 wherein there are two health sensors that independently measure at least one of the following physiological signals: blood pressure, oxygen levels, electrical conduction, pulse, respiration, heart rate, heart rhythm, activity levels, metabolic rate, glucose levels, and pulse-ox.

6. The device of claim 5 wherein there are three health sensors that independently measure at least one of the following physiological signals: blood pressure, oxygen levels, electrical conduction, pulse, respiration, heart rate, heart rhythm, activity levels, metabolic rate, glucose levels, and pulse-ox.

7. A health monitoring device comprising:
a housing having a top surface and bottom surface;
an adhesive layer adhered to the bottom surface of the housing;
a conductive adhesive hydrogel layer, which is adhered to the adhesive layer;
a battery capable of powering the health monitoring device and contained within the conductive adhesive hydrogel layer;
a GPS system capable of providing GPS information and contained within the conductive adhesive hydrogel layer;
a temperature sensor capable of measuring temperature information and contained within the conductive adhesive hydrogel layer;
at least one health sensor capable of measuring physiological signals and contained within the conductive adhesive hydrogel layer;
a storage memory contained within the conductive adhesive hydrogel layer, wherein the storage memory is in communication with the GPS system, the temperature system, at least one health sensor;
a communication chip capable of communicating with an external device and contained within the conductive adhesive hydrogel layer, wherein the communication chip is in communication with the storage memory; and,
a haptic capable of signaling a user of the health monitoring device and contained within the conductive adhesive hydrogel layer.

8. The device of claim 7 further comprising a bottom layer below the conductive adhesive hydrogel layer and wherein the bottom layer encloses health monitor device and includes an adhesive layer for attachment.

9. The device of claim 7 wherein the battery is rechargeable.

10. The device of claim 7 wherein the at least one health sensor measures at least one of the following physiological signals: blood pressure, oxygen levels, electrical conduction, pulse, respiration, heart rate, heart rhythm, activity levels, metabolic rate, glucose levels, and pulse-ox.

11. The device of claim 10 wherein there are two health sensors that independently measure at least one of the following physiological signals: blood pressure, oxygen levels, electrical conduction, pulse, respiration, heart rate, heart rhythm, activity levels, metabolic rate, glucose levels, and pulse-ox.

12. The device of claim 11 wherein there are three health sensors that independently measure at least one of the following physiological signals: blood pressure, oxygen levels, electrical conduction, pulse, respiration, heart rate, heart rhythm, activity levels, metabolic rate, glucose levels, and pulse-ox.

13. A system for monitoring the training of an athlete wherein the system comprises:
a health monitoring device comprising:
a housing having a top surface and bottom surface;
an adhesive layer adhered to the bottom surface of the housing;
a conductive adhesive hydrogel layer, which is adhered to the adhesive layer;
a battery capable of powering the health monitoring device and contained within the conductive adhesive hydrogel layer;
a GPS system capable of providing GPS information and contained within the conductive adhesive hydrogel layer;
a temperature sensor capable of measuring temperature information and contained within the conductive adhesive hydrogel layer;
at least one health sensor capable of measuring physiological signals and contained within the conductive adhesive hydrogel layer;
a processor capable of processing and storing information and contained within the conductive adhesive hydrogel layer, wherein the processor is in communication with the GPS system, the temperature system, and the at least one health sensor;
a storage memory contained within the conductive adhesive hydrogel layer, wherein the storage memory is in communication with the processor;
a communication chip capable of communicating with an external device and contained within the conductive adhesive hydrogel layer, wherein the communication chip is in communication with the storage memory;
a haptic capable of signaling a user of the health monitoring device and contained within the conductive adhesive hydrogel layer; and,
a remote display device comprising:
a display that is capable of displaying all of the health information that it receives from a health monitoring device;
a processor that is capable of running an application for processing physiological signals received externally;
a communication chip capable of communicating with an external device; and,
a haptic singular that is capable of communicating with an external haptic.

14. The device of claim 13 further comprising a bottom layer below the conductive adhesive hydrogel layer and wherein the bottom layer encloses health monitor device and includes an adhesive layer for attachment.

15. The device of claim 13 wherein the battery is rechargeable.

16. The device of claim 13 wherein the at least one health sensor measures at least one of the following physiological signals: blood pressure, oxygen levels, electrical conduction, pulse, respiration, heart rate, heart rhythm, activity levels, metabolic rate, glucose levels, and pulse-ox.

17. The device of claim 16 wherein there are two health sensors that independently measure at least one of the following physiological signals: blood pressure, oxygen levels, electrical conduction, pulse, respiration, heart rate, heart rhythm, activity levels, metabolic rate, glucose levels, and pulse-ox.

18. The device of claim 17 wherein there are three health sensors that independently measure at least one of the following physiological signals: blood pressure, oxygen levels, electrical conduction, pulse, respiration, heart rate, heart rhythm, activity levels, metabolic rate, glucose levels, and pulse-ox.
